(19) United States
(12) Patent Application Publication
(10) Pub. No.: US 2010/0276305 A1
(43) Pub. Date: Nov. 4, 2010

(54) DUCK-A-DENT

(76) Inventor: Ronnie Marcia Haber, New York, NY (US)

Correspondence Address: URSULA B. DAY, ESQ., 708 Third Avenue, SUITE 1501, NEW YORK, NY, 10017, US

(21) Appl. No.: 12/435,113

(22) Filed: May 4, 2009

(51) Int. Cl. A61B 19/02

(52) U.S. Cl. 206/63.5

(57) ABSTRACT

A box for hiding denture and denture related material is proposed, wherein the box includes several compartments and has an outer disguise.

### BACKGROUND OF THE INVENTION

The present invention relates to a carrying or storage box and in particular to storage box for dentures that hides the dentures as well as creams used with the dentures and which are considered unsightly. The box is decorated with a “powder puff”.

There are boxes which are designed to hold dentures and denture related materials. However, these boxes had the draw-back that they are too small with too few compartments. Therefore, the new box was created to avoid these draw-backs.

### SUMMARY OF THE INVENTION

The Duck-a-Dent box was conceived be able to store all denture- and denture related materials in one space to avoid embarrassment of having dentures and denture cream out in the open. To ensure privacy, the duck-a-dent box is conceived to stay on a standard toilet tank.

The Duck-A-Dent box has various compartments into which certain items fit. For example, a rear compartment can store a tooth brush and a tube of tooth paste. There will be a space for dental wafers if wafers for adhesion are desired.

In one aspect of the invention, the box for storing dentures and denture related products includes a body divided into a number of compartments with at least one compartments for storing the dentures and adjacent compartment for denture related products; each compartment is of a dimension accommodating a respective product to be stored; separate compartment lids cover each compartment of the box; a lid to close the entire box and a disguise which is a separate piece and can be in the form of a powder puff. The disguise can be placed on the closed lid, but is also for placing on top of the compartments when the lid of the box is open, for example, to disguise the purpose of the box and to disguise its content when the box lid is in the open position. The disguise can be detachably connected to the box lid, for example by a hook and loop attachment system.

Duck-A-Dent is an intimate denture kit and the box covered with a lid can also be used for traveling. In any event, the box affords a certain amount of privacy, in that the owner no longer has to fear hiding the dentures from grandchildren, lovers or friends when using the bathroom where the dentures may be sitting in a cup.

The Duck-A-dent box stores a two months supply of cleanser tablets in two side compartments. The cleansing tablets that come in sheets of push-out tablets are individually separated but strips of the tablets can be torn from the sheet and fitted into the side compartments of the box. It can house at least 60 tabs or a 2 months supply of denture cleanser.

The box can also be used as a make-up box. In each compartment particular lipsticks and/or pencils can be stored; clips rings and other jewelry can also be stored. The powder puff can be fasted to the cover or can be removed. All measurements are approximate.

### BRIEF DESCRIPTION OF THE DRAWINGS

Other features and advantages of the present invention will be more readily apparent upon reading the following description of currently preferred exemplified embodiments of the invention with reference to the accompanying drawing, in which:

FIG. 1 is a central cross section of a side view of the Duck-A-Dent box for storing articles;

FIG. 2 is a cross section of a side elevational view along axis AA showing the false bottom and the powder puff on top of the compartments;

FIG. 3 is a rear elevational view of the Duck-A-Dent box;

FIG. 4 is a top elevational view of the Duck-A-Dent box with individual compartment lids;

FIG. 5 is a front elevational view of the Duck-A-Dent box with lid in closed position;

FIG. 6 is another side elevational view of the Duck-A-Dent box with the lid in closed position and the powder puff attached to the lid;

FIG. 7 is a top view of the open Duck-A-Dent box shown without a lid;

FIG. 8 is a top view of the powder puff which is attachable to the lid of the Duck-A-Dent box;

FIG. 9 is a front, side and top perspective view of the Duck-A-Dent box in a lid open position showing the powder puff cover;

### DETAILED DESCRIPTION OF PREFERRED EMBODIMENTS

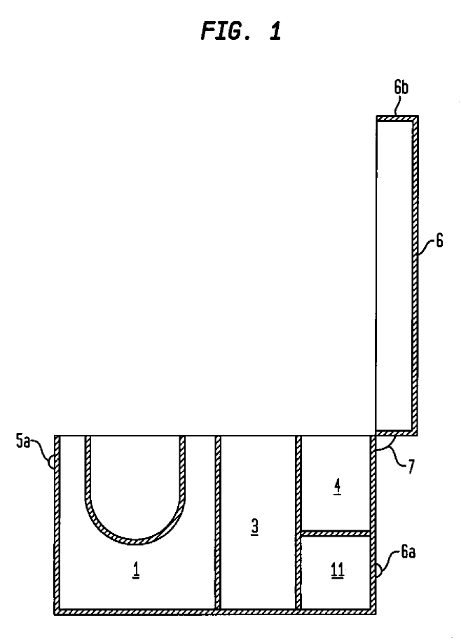

FIG. 1 shows the compartments of the Duck-A-Dent box. The box is approximately 8½ inches wide, 8 inches long and 4 inches deep.

The compartment 1 is for the denture cup with thumb cut-outs 1a are 1½ inches long and 1 inch wide. The side compartments 2 and 2a for the dental wafers is approx. 3 inches wide and 4 and ¾ long. The drawer 11 is approximately 2 inches wide 8 and ¼ inches long and 1 and ½ inches deep. The compartment 4 is for tooth brush and tooth paste and approximately 2 inches long 9 inches wide and 1½ inches deep. The lid 6 has an approximate length of 7 and ½ inches and is 1 inch deep. The hinges 7 are located approximately 1 inch from each edge. The lid skirt 6b is approximately 1 inch deep. The 1 inch pull-tab 6a is located approximately 4 inches inward form each end and has approximately a ⅝ inch center protrusion. With pull-tab 6a, a drawer 11 can be opened. Front protrusion 5a is approximately ¾ inches from the top edge of the box with a latch to snap over the 5a protrusion.

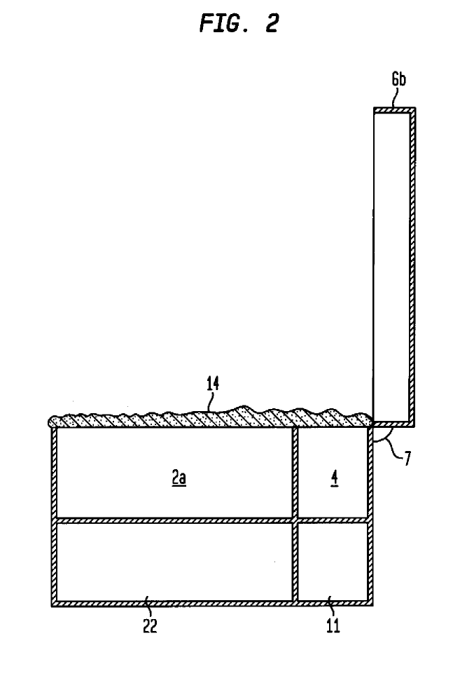

FIG. 2 shows a side view section along the axis M showing the hollow space 22 underneath compartment 2a as a false bottom with the following approximate dimensions: length 6 inches, width 2 inches and depth 1¼ inches. The compartment for the cleanser tabs is 6 inches long, 2 inches wide and 1¾ inches deep. The compartment 4 is for the Fixodent grip, brush and dentures and has dimensions 1¾ inches long; 6 inches wide and 1¾ inches deep.

The hinges are designated by 7 and are approximately 1 inch long and located approximately 1 inch from the end. The lid skirt 6b is approximately 7½ inches long and 1 inch high with the sides skirts approximately the same dimension. Also shown in FIG. 2 is the powder puff 14 placed on top of the open box, so the content is hidden by the powder puff.

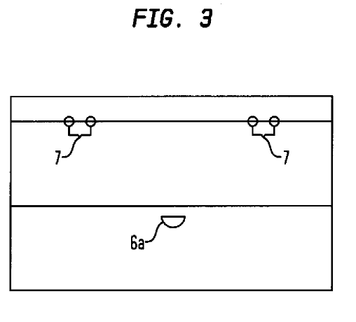

The rear view in FIG. 3 shows the hinges 7 and the drawer 11 with an approximately length of 2 inches, width of 8¼ inches and depth of 1½ inches. The rear wall dimension of the Duck-A-Dent box is approximately 1½ long and 9 inches wide. The hinges 7 are approx. 1 inch from each end and are 1 inch wide on each side.

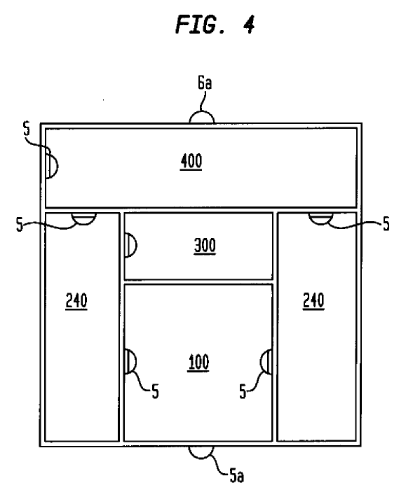

FIG. 4 is a top view of the Duck-A-Dent box with the side compartments 2 and 2a (dental tabs) being approx. 2 inches wide, 6 inches long and 3 inches deep. The dental wafer compartment 3 is approx. 3 inch long and 4½ inches wide and 3 inches deep. The denture compartment 1 has an approximate length of 3⅝ inches, width 4¾ inches and depth 3 inches. Compartment 4 for tooth paste and tooth brushes has an approx. dimension of 3⅝ length, 4¾ width and 3 inches depth.

Compartment cover 400 is the cover for compartment 4 which can store elongate articles such as tooth paste and denture cream with a length of approx. 6 inches wide, 2 long with a depth of 1 and ¾ inches. The front closure 5a is located about ¾ of an inch from the top of the box with a latch to snap over a protrusion. Pull tab 6a for compartment 4 is approx. 4 inches from the end on both sides. Thumb cut-outs 1a are 1½ inches wide and 1 inch long.

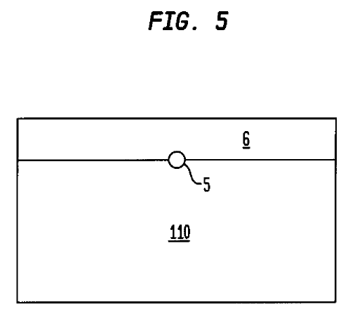

FIG. 5 shows the front wall 110 of the box with a length of 8 inches and a width of 9 inches. Knob closure 5a is located about ¾ down from the top with a latch in center of the top (not shown) to snap over the knob 5a. Skirt 6b is the front section of the lid 6 which is 1 inch high and 9 inches wide.

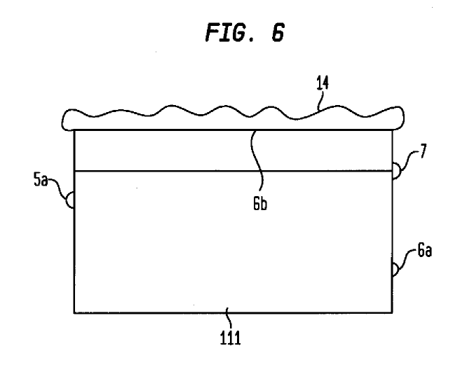

FIG. 6 is a side view of the Duck-A-Dent box with the side panel 111 being approx. 8 inches long and 9 inches wide. The skirt portion 6b of the lid is approx. 1 inch high and 7½ inches wide. The front knob closure 5a is located about ¾ inches downward from the lid and is being latched by a snap down latch (not shown here) over a protrusion. Pull tab 6a is for a drawer 11 and is 4 inch in from each end with a width of ⅝ inches.

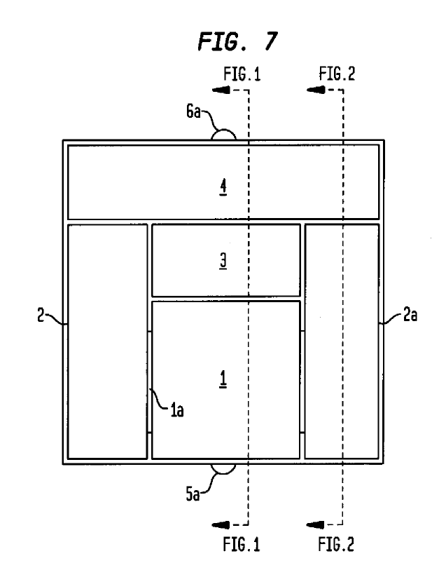

FIG. 7 shows the Duck-A-Dent box in open lid position, wherein each of the compartments is covered by individual covers lodged over each of the compartments. Thumb cut-outs 5 on each of the covers for easy lift of the covers. The cover of the denture compartment 100 is approximately 4¾ inches wide, 3⅝ inches long and 3 inches deep, with the cover of the dental wafer compartment cover 300 being approximately 3 inches long and 4¾ inches wide. Compartment covers 240 are for storing cleansing tabs are approximately 6 inches in length, 2 inches in width and 1¾ inches deep.

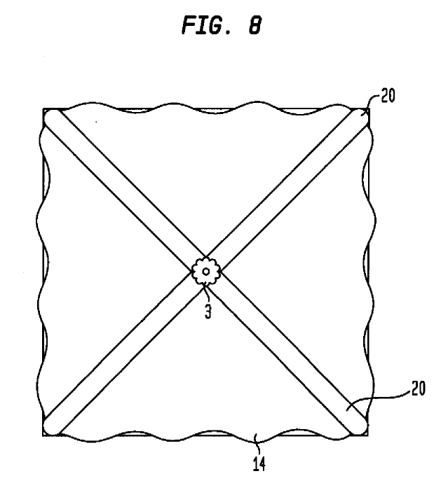

FIG. 8 is a top view of the powder puff 14 with approximate dimensions of 6¾ inches long and 8¼ inches wide. Satin ribbons 20 are crisscrossing the powder puff in the diagonal here with dimensions of 10¼ inches long and ¾ inches wide. A rose button 33 for decoration here is at the center where the ribbons cross with a dimension of ½ inch in diameter.

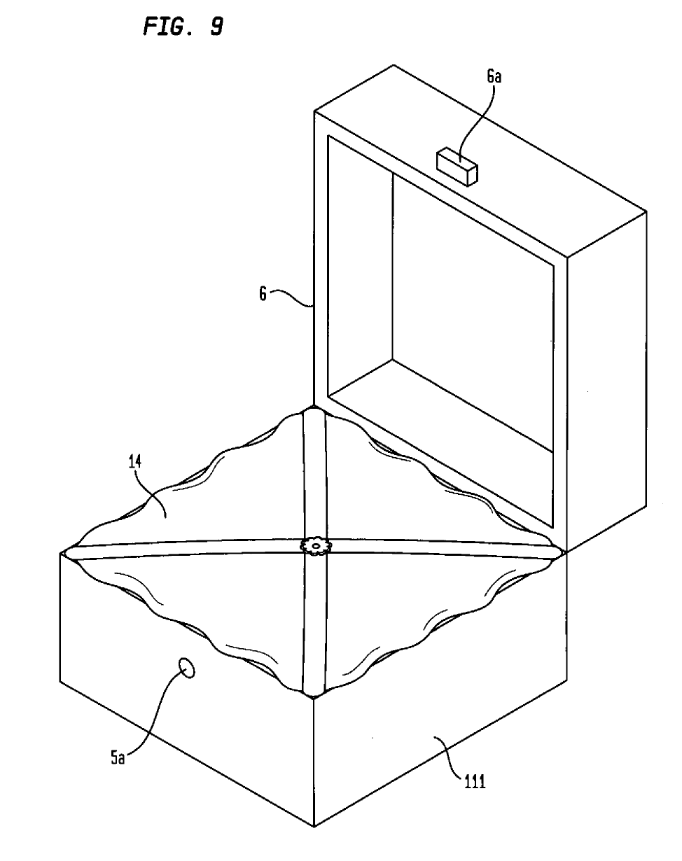

In FIG. 9 the Duck-a-Dent box is shown in perspective view with an open lid. The powder puff 14 is here placed on the opening of the box whereby the content of the box is hidden. The powder puff can be placed loosely on the opening or can be attached by hook and loop fastener strips. Likewise, when the powder puff is placed on the closed lid of the Duck-A-Dent box, it can be attached thereto by hook and loop fasteners.

While the invention has been illustrated and described as embodied in a denture box, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and their equivalents:

What is claimed is:

1. A box for storing dentures and denture related products, the box comprising:
a body divided into at least two compartments for storing the dentures and the denture related products; each compartment is of a dimension accommodating a respective product to be stored; and
a lid covering the box; and
a disguise attached to the lid to disguise the purpose of the box.

2. The box of claim 1, wherein there are at least 6 storage compartments.

3. The box of claim 2, wherein each compartment is provided with an individual cover.

4. The box of claim 4, wherein each compartment cover is provided with a cut-out for lifting the cover off.

5. The box of claim 2, wherein the box has pull-tabs.

6. The box of claim 1, wherein the disguise is in the form of a powder puff.

7. The box of claim 2, wherein one of the compartments is a drawer.

8. The box of claim 2, wherein at least two compartments have hollow bottoms.
